(19) United States
(12) Patent Application Publication
(10) Pub. No.: US 2009/0292225 A1
(43) Pub. Date: Nov. 26, 2009

(54) MEDICAL DEVICE INCLUDING A BRAID FOR CROSSING AN OCCLUSION IN A VESSEL

(75) Inventors: HANCUN CHEN, MAPLE GROVE, MN (US); PU ZHOU, MAPLE GROVE, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., MAPLE GROVE, MN (US)

(21) Appl. No.: 12/124,958

(22) Filed: May 21, 2008

(51) Int. Cl. A61M 25/09; B32B 37/00

(52) U.S. Cl. 600/585; 156/86

(57) ABSTRACT

The invention provides design, material, manufacturing method, and use alternatives for medical devices. An example medical guidewire includes an elongate core member including a proximal region and a distal region, a braid member disposed about at least a portion of the distal region of the elongate core member, a polymer member disposed between at least a portion of the elongate core member and the braid member, and a polymer sleeve member disposed about at least a portion of the braid member. The proximal region of the core member is free of the braid member.

### FIELD OF THE INVENTION

The invention relates generally to medical devices. More specifically, the invention relates to intracorporal medical device, such as a guidewire, catheter, or the like, including structure for crossing an occlusion in a vessel or a patient.

### BACKGROUND

The use of intravascular medical devices has become an effective method for treating many types of vascular disease. In general, one or more suitable intravascular devices are inserted into the vascular system of the patient and navigated through the vasculature to a desired target site. Using this method, virtually any target site in the patient's vascular system may be accessed, including the coronary, cerebral, and peripheral vasculature. Examples of therapeutic purposes for intravascular devices include percutaneous transluminal angioplasty (PTA) and percutaneous transluminal coronary angioplasty (PTCA).

When in use, intravascular devices, such as a guidewire, may enter the patient's vasculature at a convenient location and then can be urged to a target region in the anatomy. The path taken within the anatomy of a patient may be very tortuous, and as such, it may be desirable to combine a number of performance features in the intravascular device. For example, it is sometimes desirable that the device have a relatively high level of pushability and torqueability, particularly near its proximal end. It is also sometimes desirable that a device be relatively flexible, particularly near its distal end, for example, to aid in steering.

In addition, medical devices, such as a guidewire, catheter, or the like, will sometimes confront an occlusion, such as a lesion and/or stenosis when passing through the vasculature to a target location. In some cases, the occlusion may completely block the vessel as is the case with a chronic total occlusion. The success of the procedure often depends on the ability to insert the medical device through the occlusion.

A number of different elongated medical device structures, assemblies, and methods are known, each having certain advantages and disadvantages. However, there is an ongoing need to provide alternative elongated medical device structures, assemblies, and methods. In particular, there is an ongoing need to provide alternative medical devices including structure or assemblies configured to aid in crossing an occlusion in a vessel of a patient, and methods of making and using such structures and/or assemblies.

### BRIEF SUMMARY

The invention provides design, material, manufacturing method, and use alternatives for medical devices. An example medical device includes an elongate core member including a proximal region and a distal region, a braid member disposed about at least a portion of the distal region of the elongate core member, a polymer member disposed between at least a portion of the elongate core member and the braid member, and a polymer sleeve member disposed about at least a portion of the braid member. In the example medical device, the proximal region of the core member may be free of the braid member. The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partially cut-away perspective view of a guidewire in accordance with one illustrative embodiment;

FIG. 2 is a partial cross-sectional view of the illustrative guidewire of FIG. 1;

FIG. 3 is a partial cross-sectional view of an alternative guidewire embodiment;

FIG. 4 is a partial cross-sectional view of a vessel including an occlusion disposed therein with the illustrative guidewire of FIG. 1 disposed within the vessel and being advanced toward the occlusion;

FIG. 5 is a view similar to that shown in FIG. 4, but with the distal section of the guidewire engaging the occlusion; and

FIG. 6 is a view similar to that shown in FIG. 4, but with the distal section of the guidewire extending through the occlusion.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

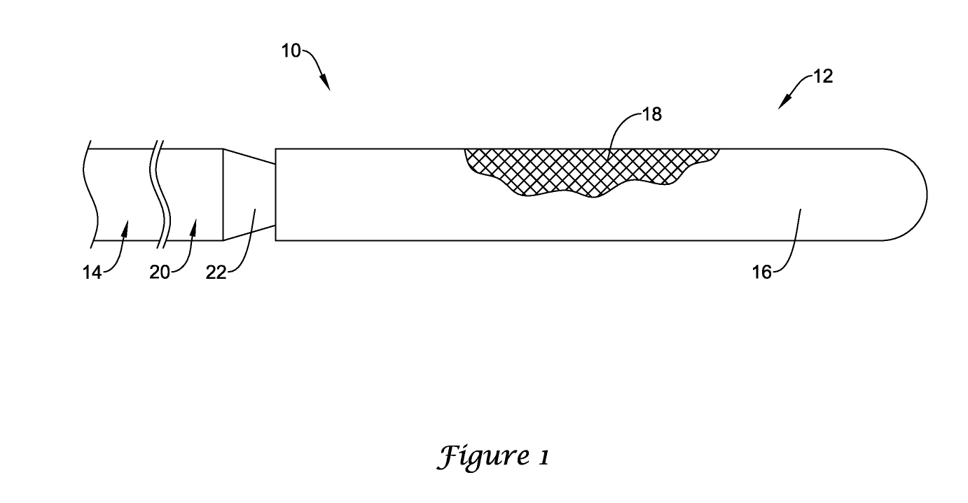

Referring now to FIG. 1, which is a partially cut-away perspective view of a medical device in accordance with one illustrative embodiment. In the embodiment shown, the medical device is in the form of a guidewire 10. In one case, the guidewire 10 may be a crossing wire that can be used to aid in crossing an occlusion in a vessel of a patient, as will be discussed in more detail below. In the illustrative embodiment, guidewire 10 can include a proximal region 14 and a distal region 12 having a distal end. As used herein, the proximal region 14 and the distal region 12 may generically refer to any two adjacent guidewire sections along any portion of the guidewire 10.

In the illustrative embodiment, guidewire 10 may include a reinforcement member or braid 18 disposed over at least a portion of a core member 20 in the distal region 12 of the guidewire 10. In some cases, braid 18 may provide guidewire 10 with a number of desirable features, as will be described in more detail below. For example, braid 18 may help to deliver torqueability and pushability in the distal region 12 of the guidewire 10.

In the example embodiment, guidewire 10 may also include a polymer sleeve member 16 disposed about the braid 18, but this is not required in all embodiments. As illustrated, the polymer sleeve 16 only covers the distal region 12. However, it is contemplated that the polymer sleeve 16 may be disposed about any portion of the guidewire 10 and/or braid 18, as desired.

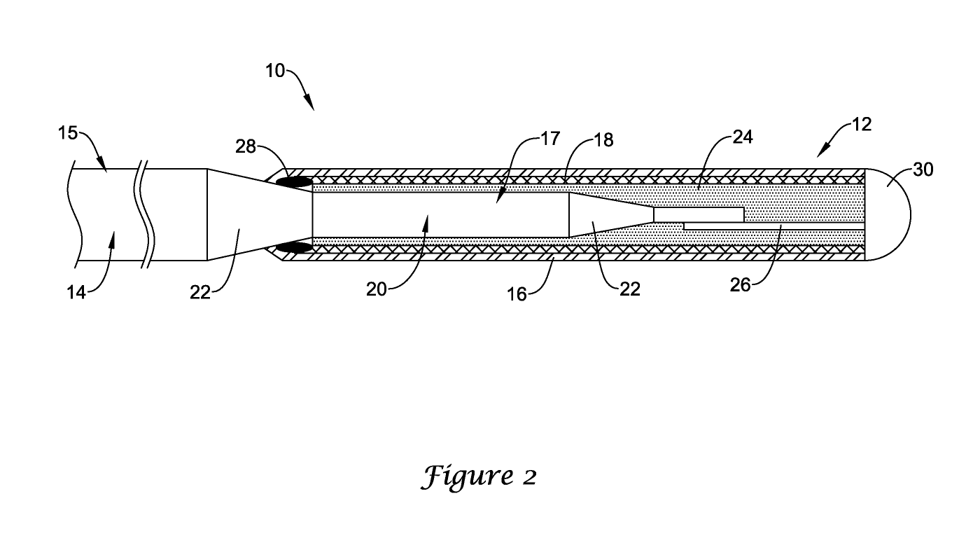

In the illustrative example, for example as shown in FIG. 2, guidewire may also include a polymer member disposed between the core member 20 and the braid 18 in at least a portion of the distal region 12 of guidewire 10. In some cases, the polymer member may be a polymer filler, a polymer tube, or any other suitable polymer member, as desired.

Referring now to FIG. 2, which is a partial cross-sectional view of the illustrative guidewire 10 of FIG. 1. In the illustrative embodiment, guidewire 10 may include elongate core member 20 including proximal region 15 and distal region 17, braid 18 disposed about at least a portion of polymer member 24, polymer member 24 disposed between at least a portion of braid 18 and core member 20, and polymer sleeve 16 disposed about at least a portion of braid 18. As illustrated, the proximal region 15 of the core member 20 may be free of the braid 18. In some cases, the proximal region 15 of the core member 20 may also be free of the polymer member 24 and polymer sleeve 16, but this is not required.

In some embodiments, the core member 20 can have a solid cross-section, for example a core wire, but in some embodiments, can have a hollow cross-section. In yet other embodiments, core member 20 can include a combination of areas having solid cross-sections and hollow cross sections. Moreover, core member 20, or portions thereof, can be made of rounded wire, flattened ribbon, or other such structures having various cross-sectional geometries. The cross-sectional geometries along the length of core member 20 can also be constant or can vary. For example, the illustrative embodiment depicts core member 20 as having a round cross-sectional shape. It can be appreciated that other cross-sectional shapes or combinations of shapes may be utilized without departing from the spirit of the invention. For example, the cross-sectional shape of core member 20 may be oval, rectangular, square, polygonal, and the like, or any suitable shape.

In some embodiments, the core member 20 may include one or more tapers or tapered portions 22, for example, to provide for desired flexibility characteristics. Such tapers can be made or exist in a linear, stepwise, curvilinear, or other suitable fashion to achieve the desired results. The angle of any such tapers can vary, depending upon the desired flexibility characteristics. The length of the taper may be selected to obtain a more (longer length) or less (shorter length) gradual transition in stiffness. For example, in the embodiment shown in FIG. 2, the core member 20 includes a plurality of tapered sections and constant diameter sections. The number, arrangement, size, and length of the narrowing and constant diameter portions can be varied to achieve the desired characteristics, such as flexibility and torque transmission characteristics. The narrowing and constant diameter portions as shown in FIG. 2 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing core wire during the grinding process. In some embodiments, core wire 14 can be centerless ground using a Royal Master HI-AC centerless grinder. Some examples of suitable grinding methods are disclosed in U.S. patent application Ser. No. 10/346,698 filed Jan. 17, 2003, which is herein incorporated by reference.

The core member 20 may include a material to impart flexibility and stiffness characteristics according to the desired application. In the illustrative embodiment, core member 20 may include a material to impart stiffness and pushability in the guidewire 10. For example, the core member 20 may include a rigid and resilient material. In such an embodiment, the core member 20 may be made from a metal, a metal alloy, a polymer, a metal-polymer composite, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®, and the like), other nickel-chromium alloys, other nickel-molybdenum alloys, other nickel-cobalt alloys, other nickel-iron alloys, other nickel-copper alloys, other nickel-tungsten or tungsten alloys, and the like; cobalt alloys, such as cobalt-chromium alloys; cobalt-chromium-molybdenum alloys (e.g., UNS: R30003 such as ELGILOY®, PHYNOX®, and the like); platinum enriched stainless steel; combinations thereof, and the like; or any other suitable material. However, this is not meant to be limiting and it is to be understood that the core member 20 may include any suitable material described herein with reference to any other guidewire component or any suitable material commonly used in medical devices, as desired.

In the illustrative embodiment, braid member 18 may be disposed over at least a portion of core member 20. In some cases, braid member 18 may be disposed over only the distal region 17 of the core member 20 and, in this example case, the proximal region 15 of core member 20 may be free of braid 18. However, in other cases, braid 18 may extend over the entire length of core member 20 or any portion thereof, as desired. For example, braid 18 may be disposed about the distal 9/10, ⅘, ¾, ⅔, ½, or ¼, of core member 20. In some embodiments, braid 18 may extend to the very distal end of core member 20, while in other embodiments, the braid 18 may extend distal of the very distal end of core member 20. In one example embodiment, the proximal end of braid 18 may be disposed distal of the proximal end of tapered portion 22 of the core member 20, if desired. As such, the length of braid 18 can vary depending upon, for example, the length of the particular device and upon the desired characteristics.

Braid member 18 may comprise a braid of interwoven strands or filaments. Braid 18 can be of any appropriate size and shape for use in the particular medical device into which it will be incorporated. In the example embodiment, braid 18 may have a generally circular cross-sectional shape, and may be appropriately sized for use in an intravascular guidewire. A broad variety of other shapes and sizes could be used, depending upon the intended use and desired characteristics of braid 18. For example, in some embodiments, braid 18 could have a flat, curved, oval, or multisided cross-sectional shape, for example, triangular, square, rectangular, pentagonal, hexagonal, and so fourth.

Furthermore, braid 18 can be formed using any suitable technique for forming the appropriate reinforcing structure. Braid 18 can be formed using a suitable number of strands or filaments. The number of strands or filaments used may often depend upon the desired characteristics of braid 18, and the patterns or techniques used to form braid 18. In some embodiments, between one and thirty-two, or even more, strands may be used in each direction.

In some embodiments, the braid member 18 can include an equal number of strands wound in each direction at the same pitch. In other words, the same number of strands may be wound in opposite directions at the same pitch. Some other embodiments may include a braid member 18 with an unequal number of strands wound in each direction. The strands in each direction may be wound at the same pitch or at differing pitches. Some examples of structures of reinforcing members can be found in U.S. patent application Ser. No. 10/346,697, filed on Jan. 17, 2003 entitled “Unbalanced Reinforcing Members for Medical Device”, which is incorporated herein by reference. The braid density may also vary widely; in some embodiments, the braid density may be as low as about 10 pic; while in other embodiments braid density may increase to the range of about 300 pic.

The strands or filaments that collectively define braid 18 may be appropriately sized and shaped depending upon the desired characteristics of braid 18 and pattern used. In some embodiments, the cross-sectional shape of the filaments can be circular, oval, flat, or multisided, for example, triangular, square, rectangular, pentagonal, hexagonal, and so fourth. In other embodiments, the filaments may be formed as ribbons.

In addition to or as an alternative to being spaced from core member 20, braid 18 may also improve torque transmission based on its material composition and configuration. For example, braid 18 may be comprised of a strong or high modulus material such as aramid (also known as poly-para-phenylene terephthalamide such as, for example, KEVLAR®, which is commercially available from DuPont). Alternatively, braid 18 or the filaments making up the braid may be made of other materials such as polymers, metals, metal alloys, or combinations thereof, for example like those materials disclosed above with reference to materials useable for the core member 20. Some examples of material for use in the braid 18 include, for example, high performance polymers, stainless steel, nickel-chromium alloy, nickel-chromium-iron alloy, cobalt alloy, tungsten, tungsten alloy, Elgiloy, MP35N, or the like, or other suitable materials. Some additional examples of suitable material include straightened super elastic (i.e., pseudoelastic) or linear elastic alloy (e.g., nickel-titanium) material, or alternatively, a polymer material, such as a high performance polymer. For example, braid 18 may include a first filament made from a combination of materials, or braid 18 may include a first filament made of a first material and a second filament made from a second material. In some embodiments, the material of braid 18 can be blended with a liquid crystal polymer (LCP). For example, the mixture can contain up to about 5% LCP. This has been found to enhance torqueability. In some other embodiments, braid 18 can include combinations of filaments or strands made up of different types of materials. Also, braid 18 can include radiopaque materials or materials that are MRI compatible. In some embodiments, the braid 18 can be made or include a radiopaque materials, as discussed herein, such as gold, platinum, tungsten, molybdenum, or the like, or alloys thereof. In some cases, tungsten and/or molybdenum may be provided in the braid 18, such as in the braid filaments, to provide the desired radiopacity. In some cases, providing the radiopacity in the braid 18 may reduce or eliminate the need from tungsten power loading in the polymer filler member 24 or the polymer sleeve member 16.

In the example embodiment, polymer filler member 24 may be disposed about at least a portion of core member 20 in distal region 17. In some cases, polymer filler member 24 may be disposed between or intermediate of at least a portion of core member 20 and at least a portion of braid member 18. In the illustrative example, filler member 24 may be made of any suitable material, such as, for example, filler member 24 may be polymeric or otherwise include a polymer. Polymers may include high performance polymers having the desired characteristics such as flexibility and torquability. Some examples of suitable polymers may include polytetrafluroethylene (PTFE), fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysufone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®, silicones, polyethylene, Marlex high-density polyethylene, linear low density polyethylene (for example REXELL®), polyolefin, polyetheretherketone (PEEK), polyimide (PI), polyetherimide (PEI), polyeter-amid, nylon, other suitable materials, or mixtures, combinations, copolymers thereof, polymer/metal composites, lubricous polymers, and the like. In some embodiments, filler member 24 can include a liquid crystal polymer (LCP) blended with other polymers to enhance torqueability. For example, the mixture can contain up to about 5% LCP. This has been found to enhance torqueability.

Filler member 24 may be formed, for example, by coating, by extrusion, co-extrusion, interrupted layer co-extrusion (ILC), fusing or bonding one or more preformed polymer segments to core member 20, or any other appropriate method. The layer may have a uniform stiffness or a gradual reduction in stiffness from the proximal end to the distal end thereof. The gradual reduction in stiffness may be continuous as by ILC or may be stepped as by fusing together separate extruded tubular segments. In some cases, filler member 24 may be impregnated with a radiopaque filler material, such as, for example, tungsten, to facilitate radiographic visualization, if desired. Those skilled in the art will recognize that these materials can vary widely without deviating from the scope of the present invention.

In the illustrative embodiment, polymer sleeve member 16 may be disposed about at least a portion of braid member 18. In some cases, polymer sleeve member 16 may be disposed over the entire length of braid member 18, while, in other embodiment, polymer sleeve member 16 may be disposed over only a portion of braid member 18. In some embodiment, polymer sleeve member 16 may extend proximally of the proximal end of braid member 18 and extend over at least a portion core member 20 and/or filler member 24. In one embodiment, polymer sleeve member 16 is disposed over essentially the entire length of core wire 20. Also, in some cases, polymer sleeve member 16 may extend distally of the distal end of braid member 18, if desired.

Sleeve member 16 may be made of any suitable material including, for example, sleeve member 16 may be polymeric or otherwise include a polymer. Polymers may include high performance polymers having the desired characteristics such as flexibility and torquability. Some examples of suitable polymers may include polytetrafluroethylene (PTFE), fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysufone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®), silicones, polyethylene, Marlex high-density polyethylene, linear low density polyethylene (for example REXELLL®), polyolefin, polyetheretherketone (PEEK), polyimide (PI), polyetherimide (PEI), polyeter-amid, nylon, other suitable materials, or mixtures, combinations, copolymers thereof, polymer/metal composites, lubricous polymers, and the like. In some embodiments sleeve member 16 can include a liquid crystal polymer (LCP) blended with other polymers to enhance torqueability. For example, the mixture can contain up to about 5% LCP. This has been found to enhance torqueability.

Polymer sleeve member 16 may be formed, for example, by coating, by extrusion, co-extrusion, interrupted layer co-extrusion (ILC), fusing or bonding one or more preformed polymer segments to core member 20 and/or braid member 18, or any other appropriate method. The layer may have a uniform stiffness or a gradual reduction in stiffness from the proximal end to the distal end thereof. The gradual reduction in stiffness may be continuous as by ILC or may be stepped as by fusing together separate extruded tubular segments. In some embodiments, polymer sleeve member 16 may be impregnated with a radiopaque filler material, such as, for example, tungsten, to facilitate radiographic visualization. Those skilled in the art will recognize that these materials can vary widely without deviating from the scope of the present invention.

In the illustrative embodiment, guidewire 10 may also include a distal tip member 30 disposed at the distal end of distal region 12 of the guidewire 10 and/or the distal end of the braid 18, polymer filler 24, and/or polymer sleeve member 16. The distal tip member 30 may be any of a broad variety of suitable structures, for example, a solder tip, a weld tip, a pre-made or pre-formed metallic or polymer structure, or the like, that is attached or joined to the distal end of the braid 18 using a suitable attachment technique. In some embodiments, the distal tip member 30 may help to secure the braid filaments together.

In some cases, distal tip member 30 may include a polymer or other polymeric material. Distal tip 30 may include the same or different polymer material as the polymer filler member 24 and/or polymer sleeve member 16, as desired. In some cases, distal tip 30 may be impregnated with a radiopaque filler material, such as, for example, tungsten, to facilitate radiographic visualization, if desired. Those skilled in the art will recognize that these materials can vary widely without deviating from the scope of the present invention. In some cases, distal tip 30 and polymer filler member 24 and polymer sleeve member 16 may be impregnated with the same or similar radiopaque filler material. Alternatively, distal tip 30 and polymer filler member 24 and polymer sleeve member 16 may be impregnated with different radiopaque filler material. In one example, distal tip 30 may include 60, 65, 70, 75, 80, 85, 90% or any other suitable percentage of tungsten loading while the polymer filler member 24 and polymer sleeve member 16 have no or minimal tungsten loading.

In this embodiment, core member 20 may have a distal end proximal of the distal tip 30. As illustrated, in the example embodiment, a shaping ribbon 26 may be provided extending between the distal tip 30 and the distal end of the core member 20, but this is not required. In this configuration, the core member 20 is not directly attached to the distal tip 30. This may allow for greater movement of the core member 20 within the braid 18 creating greater flexibility in the distal region 12 of the guidewire 10. Additionally, the incorporation of the shaping ribbon 26 may allow the distal region 12 of the guidewire 10 to be deformed or shaped by the user, as desired. While the foregoing embodiments have been shown with a shaping ribbon 26, it is not required. It is contemplated that the core member 20 may be directly coupled to the distal tip 30 or spaced from the distal tip 30, as desired.

In such an embodiment, the shaping ribbon 26 may be made from a metal, a metal alloy, a polymer, a metal-polymer composite, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®, and the like), other nickel-chromium alloys, other nickel-molybdenum alloys, other nickel-cobalt alloys, other nickel-iron alloys, other nickel-copper alloys, other nickel-tungsten or tungsten alloys, and the like; cobalt alloys, such as cobalt-chromium alloys; cobalt-chromium-molybdenum alloys (e.g., UNS: R30003 such as ELGILOY®, PHYNOX®, and the like); platinum enriched stainless steel; combinations thereof, and the like; or any other suitable material. However, this is not meant to be limiting and it is to be understood that the shaping ribbon 26 may include any suitable material described herein with reference to any other guidewire component or any suitable material commonly used in medical devices, as desired.

An illustrative method of manufacturing the illustrative guidewire 10 may include disposing the polymer filler member 24 about at least a portion of the core member 20. In some cases, the polymer filler member 24 can be disposed about the core member 20 at desired locations, such as, for example, about the distal region of the core member 20 or about at least a portion of the tapered portion 22 of core member 20, as desired. Then, in some cases, the core member 20 and polymer filler member 24 may be exposed to a heat source causing the polymer filler member 24 to bond or otherwise become secured to core member 20.

Once the polymer filler member 24 is secured to core member 20, braid 18, which in some cases, may be preformed, may be disposed about at least a portion of polymer filler member 24. Then, braid 18 may be secured to the core member 18 and/or polymer filler member 24 by suitable techniques, such as, for example, laser welding. In some cases, the braid 18 may be secured first at the proximal end of the braid 18 and then subject to a longitudinal force in the distal direction. Then, the distal end of braid 18 may be secured to the distal end of guidewire 10, such as, for example, to distal tip 30.

Next, polymer sleeve 16 may be disposed about at least a portion of braid 18. In some cases, a shrink tube may be applied over the polymer sleeve. Then, with the shrink tube applied, a heat source may be applied to the guidewire 10 causing the polymer to reflow over the braid 18 so that the polymer sleeve 16 may adhere to the polymer filler member 24 through one or more openings in braid 18.

Essentially, in at least some embodiments, braid 18 may be partially or fully embedded within polymer filler member 24. Embedding may be accomplished in a number of ways. For example, braid 18 may be placed over polymer filler member 24 and then polymer sleeve member 16 can be placed over braid 18, and then the polymer members can be melted together. In other alternative embodiments, polymer filler member 24 may include a low melting temperature polymer that flows when exposed to heat. Braid 18 can be disposed over polymer filler member 24 and a heat shrink outer polymer sleeve 16 can be disposed over braid 18 and the various structures can be thermally treated to embed braid 18. It can be appreciated that a number of other manufacturing methods may be used to embed braid 18 within polymer layers without departing from the spirit of the invention.

An alternative method may include pre-forming the braid 18, polymer filler member 24, and/or the polymer sleeve member 16 and then, disposing the pre-formed braid, polymer filler 24, and/or polymer sleeve member 16 over the core member 20.

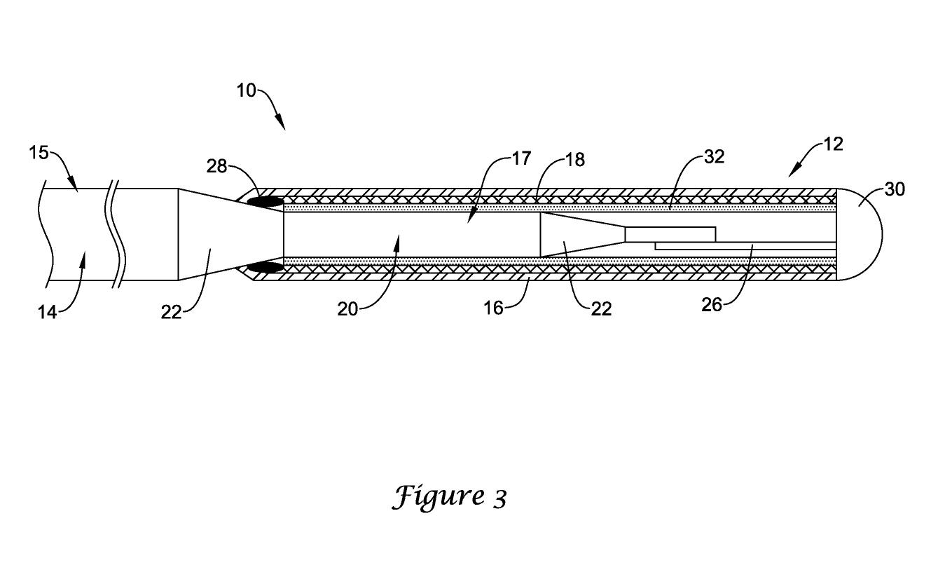

FIG. 3 is a partial cross-sectional view of an alternative guidewire embodiment. In the example embodiment, polymer filler member 24 of the embodiment of FIG. 2 may be replaced by a polymer tube 32. In the illustrative embodiment, polymer tube 32 may be disposed about at least a portion of core member 20 in distal region 17. In some cases, polymer tube 32 may be disposed intermediate core member 20 and braid member 18 in distal region 17, similar to polymer filler member of the embodiment shown in FIG. 2.

In the illustrative example, polymer tube 32 may be made of any suitable material, such as, for example, polymer tube 32 may be polymeric or otherwise include a polymer. Polymers may include high performance polymers having the desired characteristics such as flexibility and torquability. Some examples of suitable polymers may include polytetrafluroethylene (PTFE), fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysufone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®), silicones, polyethylene, Marlex high-density polyethylene, linear low density polyethylene (for example REXELLL®), polyolefin, polyetheretherketone (PEEK), polyimide (PI), polyetherimide (PEI), polyeter-amid, nylon, other suitable materials, or mixtures, combinations, copolymers thereof, polymer/metal composites, lubricous polymers, and the like. In some embodiments, polymer tube 32 can include a liquid crystal polymer (LCP) blended with other polymers to enhance torqueability. For example, the mixture can contain up to about 5% LCP. This has been found to enhance torqueability.

Polymer tube 32 may be formed, for example, by coating, by extrusion, co-extrusion, interrupted layer co-extrusion (ILC), fusing or bonding one or more preformed polymer segments to core member 20, or any other appropriate method. The layer may have a uniform stiffness or a gradual reduction in stiffness from the proximal end to the distal end thereof. The gradual reduction in stiffness may be continuous as by ILC or may be stepped as by fusing together separate extruded tubular segments. Polymer tube 32 may be impregnated with a radiopaque filler material, such as, for example, tungsten, to facilitate radiographic visualization. Those skilled in the art will recognize that these materials can vary widely without deviating from the scope of the present invention.

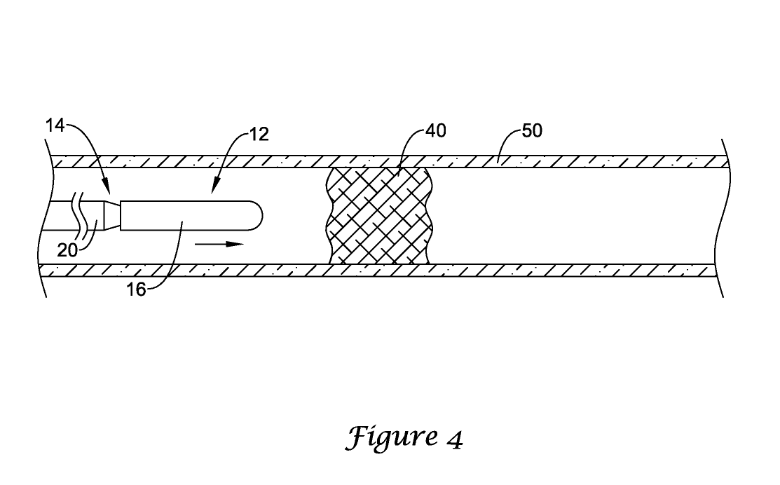

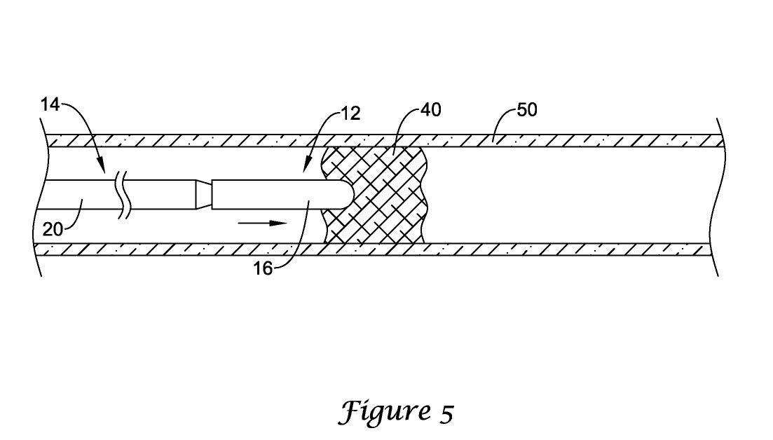

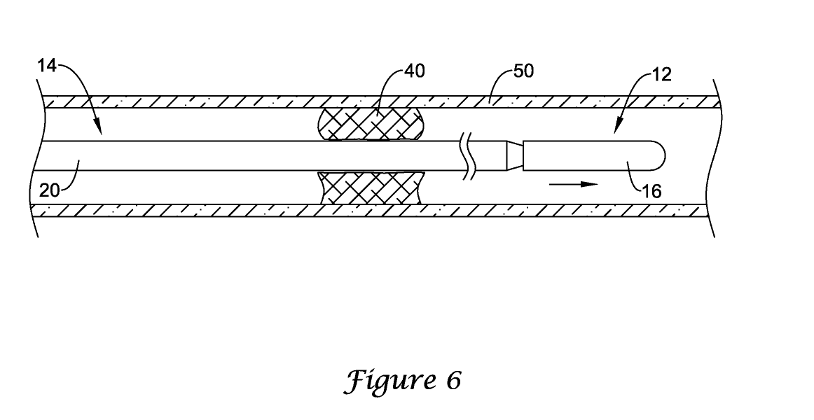

Referring now to FIGS. 4-6, which may be used in providing a discussion of one example of use of the guidewire 10. As mentioned above, the guidewire 10 may be configured to aid a user to cross an occlusion 40 in a vessel 50 of a patient. In particular, the guidewire 10 may be configured to have sufficient pushability and/or stiffness to aid crossing into and/or through occlusion 40. As shown in FIG. 4, the guidewire 10 may be advanced through the patient's vasculature, for example in a vessel 50, until it reaches an occlusion 40 within the vessel 50. As shown in FIG. 5, the distal region 12 of the guidewire 10, in particular, the distal tip, may be forced into contact with the occlusion 40. For example, the distal region 14 may be pushed slightly into the occlusion 40. Having sufficient pushability and/or stiffness, guidewire 10 may be advanced through the occlusion 40 using a sufficient force. In some cases, guidewire 10 may be rotated to assist in crossing the occlusion. Continued application of force may allow the distal section to continue to pass into the occlusion 40, and ultimately pass through the occlusion 40, as shown in FIG. 6. Once the guidewire 10 is passed through the occlusion, another device, such as a catheter, atherectomy device, distal protection device, or the like may be threaded onto the guidewire and urged distally and passed through the occlusion 40 and/or may be used to treat the occlusion 40.

In at least some embodiments, portions or all of core member 20, polymer filler member 24, sleeve member 16, polymer tube member 32, braid member 18, and/or other components that are part of or used in the device, may be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of guidewire 10 to achieve the same result.

In some embodiments, a degree of MRI compatibility is imparted into device 10. For example, to enhance compatibility with Magnetic Resonance Imaging (MRI) machines, it may be desirable to make core member 20, polymer filler member 24, sleeve member 16, polymer tube member 32, braid member 18, or other portions of the medical device 10, in a manner that would impart a degree of MRI compatibility. For example, core member 20, polymer filler member 24, sleeve member 16, polymer tube member 32, and/or braid member 18, or portions thereof, may be made of a material that does not substantially distort the image and create substantial artifacts (artifacts are gaps in the image). Certain ferromagnetic materials, for example, may not be suitable because they may create artifacts in an MRI image. Core member 20, polymer filler member 24, sleeve member 16, polymer tube member 32, and/or braid member 18, or portions thereof, may also be made from a material that the MRI machine can image. Some materials that exhibit these characteristics include, for example, tungsten, Elgiloy, MP35N, nitinol, and the like, and others.

In some embodiments, a sheath and/or coating, for example a lubricious, a hydrophilic, a protective, or other type of material may be applied over portions or all of the core member 20, polymer filler member 24, sleeve member 16, polymer tube member 32, and/or braid member 18, or other portions of device 10. Some examples of suitable coating materials may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Some coating polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. Some examples of coatings would be disposing a coating on the core member 20, the polymer sleeve member 16, and/or the braid member 18.

The length of the guidewire 10 is typically dictated by the length and flexibility characteristics desired in the final medical device. For example, proximal section 14 may have a length in the range of about 20 to about 300 centimeters or more, the distal section 12 may have a length in the range of about 3 to about 50 centimeters or more, and the medical device 10 may have a total length in the range of about 25 to about 350 centimeters or more. It can be appreciated that alterations in the length of sections and/or of the guidewire 10 as a whole can be made without departing from the spirit of the invention.

In some cases, core member 20 can be made of the same material along its length, or in some embodiments, can include portions or sections made of different materials. In some embodiments, the material used to construct core member 20 is chosen to impart varying flexibility and stiffness characteristics to different portions of core member 30. For example, the proximal region and the distal region of core member 20 may be formed of different materials, for example materials having different moduli of elasticity, resulting in a difference in flexibility. In some embodiments, the material used to construct the proximal region can be relatively stiff for pushability and torqueability, and the material used to construct the distal region can be relatively flexible by comparison for better lateral trackability and steerability. For example, the proximal region can be formed of straightened 304v stainless steel wire or ribbon and the distal region can be formed of a straightened super elastic or linear elastic alloy, for example a nickel-titanium alloy wire or ribbon.

In embodiments where different portions of core member 20 are made of different materials, the different portions can be connected using any suitable connecting techniques. For example, the different portions of core member 20 can be connected using welding (including laser welding), soldering, brazing, adhesive, or the like, or combinations thereof. Additionally, some embodiments can include one or more mechanical connectors or connector assemblies to connect the different portions of core member 20 that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. No. 09/972,276 filed on Oct. 5, 2001, Ser. No. 10/068,992 filed on Feb. 28, 2002, and Ser. No. 10/375,766 filed on Feb. 26, 2003, which are incorporated herein by reference.

It should also be understood that a broad variety of other structures and/or components may be used in the guidewire construction. Some examples of other structures that may be used in the guidewire 10 include one or more coil members, braids, shaping or safety structures, such as a shaping ribbon or wire, marker members, such as marker bands or coils, centering structures for centering the core wire within the tubular member, such as a centering ring, an extension system, for example, to effectively lengthen the guidewire for aiding in exchanging other devices, or the like, or other structures. Those of skill in the art and others will recognize that the materials, structure, and dimensions of the guidewire may be dictated primary by the desired characteristics and function of the final guidewire, and that any of a broad range of materials, structures, and dimensions can be used.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. For example, although set forth with specific reference to guidewires in some of the example embodiments shown in the Figures and discussed above, the invention may relate to virtually any medical device that may aid a user of the device in crossing an occlusion in a blood. For example, the invention may be applied to medical devices such as a balloon catheter, an atherectomy catheter, a drug delivery catheter, a stent delivery catheter, an endoscope, a fluid delivery device, other infusion or aspiration devices, delivery (i.e. implantation) devices, and the like. Thus, while the Figures and descriptions above are directed toward a guidewire, in other applications, sizes in terms of diameter, width, and length may vary widely, depending upon the desired properties of a particular device. The scope of the invention is, of course, defined in the language in which the appended claims are expressed.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A medical guidewire comprising:
an elongate core member including a proximal region and a distal region;
a braid member disposed about at least a portion of the distal region of the elongate core member;
a polymer member disposed between at least a portion of the elongate core member and the braid member; and
a polymer sleeve member disposed about at least a portion of the braid member;
wherein the braid member does not extend over the proximal region of the core member.

2. The medical guidewire of claim 1 wherein the distal region of the core member includes one or more tapers.

3. The medical guidewire of claim 2 wherein the braid member includes a proximal end and a distal end, the proximal end of the braid member being distal of the proximal most portion of the one or more tapers.

4. The medical guidewire of claim 1 wherein the braid member extends distally of a distal end of the core member.

5. The medical guidewire of claim 1 wherein the polymer member is a polymer filler member.

6. The medical guidewire of claim 1 wherein the polymer member is a polymer filler tube member.

7. The medical guidewire of claim 1 wherein the core member includes a nickel-titanium alloy.

8. The medical guidewire of claim 1 wherein the core member includes stainless steel.

9. The medical guidewire of claim 1 wherein the braid member includes a plurality of filaments, wherein at least some of the filaments include a radiopaque material.

10. The medical guidewire of claim 9 wherein the radiopaque material in at least some of the plurality of braid member filaments is tungsten.

11. The medical guidewire of claim 9 wherein the radiopaque material in at least some of the plurality of braid member filaments is molybdenum

12. The medical guidewire of claim 1 further comprising:
a distal tip disposed at the distal end of the core member, the distal end of braid member, a distal end of polymer member, and/or a distal end of polymer sleeve member; and
a shaping ribbon attached adjacent to the distal end of the core member and to the distal tip.

13. A method of making a medical guidewire, the method comprising:
providing an elongated core member having a proximal region and a distal region;
disposing a polymer member about at least a portion of the distal region of the core member;
disposing a braid member about at least a portion of the polymer member; and
disposing a polymer sleeve about at least a portion of the braid member;
wherein the braid member does not extend about the proximal region of the core member.

14. The method of claim 13 wherein the polymer member is a polymer tube member, wherein the polymer tube member, braid member, and polymer sleeve member are assembled and the assembled polymer tube member, braid member, and polymer sleeve member are disposed over the core member.

15. The method of claim 13 wherein the braid includes a plurality a filaments, wherein at least some of the plurality of filaments include a radiopaque material.

16. The method of claim 13 further comprising:
placing a heat shrink tube over the polymer sleeve member; and
heating the guidewire such that the polymer member and the polymer sleeve member adhere to each other.

17. A medical guidewire comprising:
an elongate core member having a distal region and a proximal region;
a braid member including a plurality of filaments and having a distal end, the braid member disposed about the core member in only the distal region;
a polymer filler member disposed intermediate the core member and the braid member; and
a polymer sleeve member disposed about at least a portion of the braid member.

18. The medical guidewire of claim 17 further comprising:
a distal tip disposed distal the distal end of the braid member; and
a ribbon attached to the distal tip and the distal end of the core member.

19. The medical guidewire of claim 17 wherein at least some of the plurality of filaments of the braid member include a radiopaque material.

20. A medical guidewire comprising:
an elongate core member having a distal region and a proximal region;
a braid member including a plurality of filaments and having a distal end, the braid member disposed about the core member in only the distal region;
a polymer tube member disposed intermediate the core member and the braid member; and
a polymer sleeve member disposed about at least a portion of the braid member.

21. The medical guidewire of claim 20 further comprising:
a distal tip disposed distal the distal end of the braid member; and
a ribbon attached to the distal tip and the distal end of the core member.

22. The medical guidewire of claim 20 wherein at least some of the plurality of filaments of the braid member include a radiopaque material.

23. The medical guidewire of claim 22 wherein the radiopaque material is tungsten.

24. The medical guidewire of claim 22 wherein the radiopaque material is molybdenum.
